SEQUENCE LISTING (8 sequences)

SEQ ID NO: 1 - protein, 27 aa, Artificial
  Chimeric T Helper and CTL epitope peptide
KQIINMWQEVGKAMYAKAFSPEVIPMF

SEQ ID NO: 2 - protein, 47 aa, Artificial
  Chimeric T Helper and CTL epitope peptide
YKRWIILGLNKIVRMYSNPPIPVGEIYKRWIILGLNKIVRMYSPTSI

SEQ ID NO: 3 - protein, 32 aa, Artificial
  Chimeric T Helper and CTL epitope peptide
DRVIEVVQGAYRAILVGFPVRPQVPLRPMTYK

SEQ ID NO: 4 - protein, 36 aa, Artificial
  Chimeric T Helper and CTL epitope peptide
KQIINMWQVVGKAMYAGQMVHQAISPRTLNAWVKVV

SEQ ID NO: 5 - protein, 45 aa, Artificial
  Chimeric T Helper and CTL epitope peptide
KQIINMWQVVGKAMYAEPFRDYVDRFYKTLRAEQASQEVKNWMTE

SEQ ID NO: 6 - protein, 35 aa, Artificial
  Chimeric T Helper and CTL epitope peptide
KQIINMWQVVGKAMYAKIRLRPGGKKKYKLKHIVW

SEQ ID NO: 7 - protein, 39 aa, Artificial
  Chimeric T Helper and CTL epitope peptide
KQIINMWQVVGKAMYATGSEELRSLYNTVATLYCVHQRI

SEQ ID NO: 8 - protein, 48 aa, Artificial
  Chimeric T Helper and CTL epitope peptide
KQIINMWQVVGKAMYASPAIFQSSMTKILEPFRKQNPDIVIYQYMDDL

(19) United States
(12) Patent Application Publication
(10) Pub. No.: US 2008/0187554 A1
(43) Pub. Date: Aug. 7, 2008

(54) Process for producing stable HIV Th-CTL peptides

(75) Inventors: Lakshmi Khandke, Nanuet, NY (US); Ranjit Sarpal, Monroe Twp., NJ (US); Xudong Cindy Yang, Tappan, NY (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 11/592,404

(22) Filed: Nov. 3, 2006

Related U.S. Application Data
(60) Provisional application No. 60/733,979, filed on Nov. 3, 2005.

(51) Int. Cl. A61K 39/21; C07K 14/155; A61P 31/18

(52) U.S. Cl. 424/188.1; 530/324

(57) ABSTRACT

A process for producing a stable formulation of hydrophobic HIV Th/CTL peptides first requires the preparation of a formulation buffer containing 12.5 mM succinic acid and 3% mannitol, and adjusting the pH of the formulation buffer to 1.7 to 2.1. The hydrophobic peptides are then dissolved in the formulation buffer, filtered and lyophilized. Prior to use, the lyophilized peptides are reconstituted with 12.5 mM sodium succinate, and then mixed with at least one adjuvant to produce an immunogenic composition that is stable at 2-8° C. for at least three hours.

This application claims the benefit of U.S. Provisional Application No. 60/733,979 filed Nov. 3, 2005.

### FIELD OF THE INVENTION

The present invention relates to a formulation buffer and the use of this buffer in a process for generating a stable formulation of HIV Th/CTL peptides for viral immunogenic composition production.

### BACKGROUND OF THE INVENTION

Many studies have been carried out to develop a prophylactic or therapeutic vaccine against HIV, and many antigens have been proposed for this purpose. By way of example, a tetravalent multi-epitope Th/CTL peptide (Th/CTL A, B, C and J) composition has been developed for use as a prophylactic or therapeutic vaccine against HIV-1. Each peptide (27-47 amino acids) of the tetravalent composition contains one of four different CTL “hot spots” from gag or nef and one of four different HIV-derived T-helper epitopes from envor gag. The peptide sequences are as follows:

A-Th/A-CTL (Peptide A):

(SEQ ID NO:1)

H-Lys-Gln-Ile-Ile-Asn-Met-Trp-Gln-Glu-Val-Gly-Lys-
Ala-Met-Tyr-Ala-Lys-Ala-Phe-Ser-Pro-Glu-Val-Ile-
Pro-Met-Phe-OH
B-Th/B-CTL (PeptideB):

(SEQ ID NO:2)

H-Tyr-Lys-Arg-Trp-Ile-Ile-Leu-Gly-Leu-Asn-Lys-Ile-
Val-Arg-Met-Tyr-Ser-Asn-Pro-Pro-Ile-Pro-Val-Gly-
Glu-Ile-Tyr-Lys-Arg-Trp-Ile-Ile-Leu-Gly-Leu-Asn-
Lys-Ile-Val-Arg-Met-Tyr-Ser-Pro-Thr-Ser-Ile-OH
C-Th/C-CTL (Peptide C):

(SEQ ID NO:3)

H-Asp-Arg-Val-Ile-Glu-Val-Val-Gln-Gly-Ala-Tyr-Arg-
Ala-Ile-Arg-Val-Gly-Phe-Pro-Val-Arg-Pro-Gln-Val-
Pro-Leu-Arg-Met-Thr-Tyr-Lys-OH
A*-Th/J-CTL (Peptide J):

(SEQ ID NO:4)

H-Lys-Gln-Ile-Ile-Asn-Met-Trp-Gln-Val-Val-Gly-Lys-
Ala-Met-Tyr-Ala-Gly-Gln-Met-Val-His-Gln-Ala-Ile-
Ser-Pro-Arg-Thr-Leu-Asn-Ala-Trp-Val-Lys-Val-Val-OH

These peptides however, disclosed in WO 01/56355 and incorporated herein by reference, are very hydrophobic in nature and have limited solubility in aqueous systems. Thus, there is a need for a process for dissolving each of these hydrophobic Th/CTL peptides so that they can be formulated into a stable composition for use against HIV.

### SUMMARY OF THE INVENTION

To meet this need, the present invention provides a process for solubilizing hydrophobic HIV Th/CTL peptides, comprising the steps of:
(a) preparing an aqueous formulation buffer comprising 12.5 mM succinic acid and 3% mannitol;
(b) adjusting the pH of the formulation buffer to 1.7 to 2.1; and
(c) dissolving at least one hydrophobic HIV Th/CTL peptide in the formulation buffer.

In one embodiment, a mixture of at least four hydrophobic HIV Th/CTL peptides is dissolved in the formulation buffer. This peptide mixture comprises HIV A-Th/A-CTL (SEQ ID NO: 1), B-Th/B-CTL (SEQ ID NO: 2), C-Th/C-CTL (SEQ ID NO: 3) and A*-Th/J-CTL (SEQ ID NO: 4).

In another embodiment, a mixture of at least eight hydrophobic HIV Th/CTL peptides is dissolved in the formulation buffer. This peptide mixture comprises HIV A-Th/A-CTL (SEQ ID NO: 1), B-Th/B-CTL (SEQ ID NO: 2), C-Th/C-CTL (SEQ ID NO: 3), A*Th/J-CTL (SEQ ID NO: 4), A*-Th/L-CTL (SEQ ID NO: 5), A*-Th/M1-CTL (SEQ ID NO: 6), A*-Th/M2-CTL (SEQ ID NO: 7) and A*-Th/R-CTL (SEQ ID NO: 8). See Table 1.

TABLE 1
Amino Acid Sequences of HIV Th/CTL Peptides

| Name of | | SEQ |
|---|---|---|
| Peptide | Amino Acid Sequence | ID NO. |
| A-Th/A-CTL | KQIINMWQEVGKAMYA - | 1 |
| | KAFSPEVIPMF | |
| B-Th/B-CTL | YKRWIILGLNKIVRMYS - | 2 |
| | NPPIPVGEIYKRWIILGLNKIVRMYSPTSI | |
| C-Th/C-CTL | DRVIEVVQGAYRAIL - | 3 |
| | VGFPVRPQVPLRPMTYK | |
| A*-Th/J-CTL | KQIINMWQVVGKAMYA - | 4 |
| | GQMVHQAISPRTLNAWVKVV | |
| A*-Th/L-CTL | KQIINMWQVVGKAMYA - | 5 |
| | EPFRDYVDRFYKTLRAEQASQEVKNWMTE | |
| A*-Th/M1-CTL | KQIINMWQVVGKAMYA - | 6 |
| | KIRLRPGGKKKYKLKHIVW | |
| A*-Th/M2-CTL | KQIINMWQVVGKAMYA - | 7 |
| | TGSEELRSLYNTVATLYCVHQRI | |
| A*-Th/R-CTL | KQIINMWQVVGKAMYA - | 8 |
| | SPAIFQSSMTKILEPFRKQNPDIVIYQYMDD | |
| | L | |

The present invention further provides a process for preparing a stable formulation of hydrophobic HIV Th/CTL peptides, comprising the steps of:
(a) preparing an aqueous formulation buffer comprising 12.5 mM succinic acid and 3% mannitol, and adjusting the pH of the formulation buffer to 1.7 to 2.1;
(b) dissolving a mixture of hydrophobic HIV Th/CTL peptides in the formulation buffer;
(c) filtering the peptide formulation obtained in step (b); and
(d) lyophilizing the filtrate,
   wherein the lyophilized HIV Th/CTL peptides are stable at 2-8° C. for at least 24 months.

In one embodiment, the mixture of hydrophobic peptides comprises HIV A-Th/A-CTL (SEQ ID NO:1), B-Th/B-CTL (SEQ ID NO:2), C-Th/C-CTL (SEQ ID NO:3) and A*-Th/J-CTL (SEQ ID NO:4).

In another embodiment, a mixture of at least eight hydrophobic HIV Th/CTL peptides is dissolved in the formulation buffer. This peptide mixture comprises HIV A-Th/A-CTL (SEQ ID NO:1), B-Th/B-CTL (SEQ ID NO:2), C-Th/C-CTL (SEQ ID NO:3), A*Th/J-CTL (SEQ ID NO:4), A*-Th/L-CTL (SEQ ID NO:5), A*-Th/M1-CTL (SEQ ID NO:6), A*-Th/M2-CTL (SEQ ID NO:7) and A*-Th/R-CTL (SEQ ID NO:8).

The present invention also provides a process for preparing an immunogenic composition for the prevention or treatment of HIV, comprising: reconstituting the lyophilized HIV Th/CTL peptides obtained above with sodium succinate prior to use, wherein the pH after reconstitution is 4.2 to 5.2, and mixing the reconstituted peptides with at least one adjuvant to produce an immunogenic composition that is stable at 2-8° C. for at least three hours. The adjuvant can be GM-CSF, RC 529-SE, or a combination thereof.

The present invention also provides a lyophilized composition comprising hydrophobic HIV Th/CTL peptides that are produced by the process described above, wherein the peptides are stable at 2-8° C. for at least 24 months.

### BRIEF DESCRIPTION OF THE DRAWINGS

FIG. 1 shows the stability of the pre-lyophilized HIV peptide formulation of 1 mg/mL of each peptide held at 2-8° C. measured by RP-HPLC, Lot L22863-115;

FIG. 2 shows the stability of the pre-lyophilized monovalent HIV peptides at 1 mg/mL held at 2-8° C., Lot L22863-140 A, B, C, J;

FIG. 3 shows the stability of the lyophilized HIV peptide composition held at 2-8° C. measured by RP-HPLC, Lot L22863-115;

FIG. 4 shows the stability of the lyophilized HIV tetravalent peptide composition at 187.5 μg/mL held at 2-8° C. for 12 months tested by RP-HPLC, Lot L24282-72.1;

FIG. 5 shows the stability of the lyophilized HIV tetravalent peptide composition at 625 μg/mL held at 2-8° C. for 12 months tested by RP-HPLC, Lot L24282-72.2;

FIG. 6 shows the stability of the lyophilized HIV peptide composition held at 40° C. measured by RP-HPLC, Lot L22863-115;

FIG. 7 shows the stability of the lyophilized HIV tetravalent peptide composition at 187.5 μg/mL held at 40° C. for 12 months tested by RP-HPLC, Lot L24282-72.1;

FIG. 8 shows the stability of the lyophilized HIV tetravalent peptide composition at 625 μg/mL held at 40° C. for 12 months tested by RP-HPLC, Lot L24282-72.2;

FIG. 9 shows the fluorescence light scattering (FLS) of the lyophilized HIV peptide composition after reconstitution stored at 2-8° C., Lot L22863-115;

FIG. 10 shows the FLS of the lyophilized HIV peptide composition after reconstitution stored at 40° C., Lot L22863-115;

FIG. 11 shows the peptide fluorescence of the lyophilized HIV peptide composition after reconstitution stored at 2-8° C., Lot L22863-115;

FIG. 12 shows the peptide fluorescence of the lyophilized HIV peptide composition after reconstitution stored at 40° C., Lot L22863-115; and

FIG. 13 shows the stability of the lyophilized monovalent HIV peptides after reconstitution measured by FLS, Lot L22863-140 A, B, C, J.

### DETAILED DESCRIPTION OF THE INVENTION

Formulation of the HIV-Th/CTL-peptides presented a number of challenges. The peptides are very hydrophobic in nature. They exhibit poor solubility in common aqueous buffers, such as phosphate buffered saline (PBS), Tris, and citrate at a pH of 5 to 8 and a concentration of 1 mg/mL or higher; in co-solvents such as polyethylene glycol (PEG), ethanol, and propylene glycol; and in disperse systems, such as vegetable oils, Triton X-100, Tween 80, and encapsin. The formulation consists of a mixture of multiple Th/CTL peptides and each peptide has to be compatible with one or more adjuvants. The final composition needs to be delivered at or around physiological pH, and the composition has to be stable.

### Formulation Buffer

The initial efforts were directed towards getting the hydrophobic peptides into solution. Based on the solubility and stability studies of a wide range of buffers, co-solvents, surfactants and pHs, it was observed that succinic acid at pH 2 provided the optimum conditions for keeping the peptides soluble and stable up to 12 weeks at 2-8° C. Thus, each of the HIV Th/CTL peptides is formulated in 12.5 mM succinic acid, 3% mannitol at pH 1.7-2.1 (target pH 1.9) in water for injection (WFI).

### Peptide Composition

The liquid formulated peptide mixture is most stable at pH 1.9±0.2, but begins to precipitate after several months of storage at refrigerated temperatures. Therefore, the HIV Th/CTL peptide composition is lyophilized to enhance its stability. The pH is then raised by reconstitution with 12.5 mM sodium succinate diluent bringing it close to the physiological range for delivery.

The thermal characterization for the lyophilization cycle was developed using differential scanning calorimetry, freeze-drying microscopy, electrical resistance, cycle time and product attributes, which include cake cosmetics, reconstitution time, residual moisture and product characteristics and stability. Studies proved mannitol to be a better bulking agent than sucrose based on thermal properties and cake appearance. Mannitol raises the melting temperature and glass temperature of the peptides, allowing for a shorter lyophilization cycle and improved stability. The reconstitution characteristics were optimized at a concentration of 3% mannitol. Surfactants are not used in the formulation because of crystallization observed in the thermal analysis.

### Lyophilization Cycle

The samples are loaded on the shelf already maintained at 5° C. and equilibrated for two hours before freezing to −40° C. The condenser temperature is <−70° C. The primary drying is conducted at −20° C. with 100 mT vacuum with inclusion of annealing and thermal treatment in the freezing phase. Secondary drying is conducted at 25° C. with 100 mT vacuum as well. The details of the lyophilization cycle are recorded in Table 2 below.

TABLE 2
Lyophilization Cycle Parameters

| Lyophilization | Ramp Rate | Temperature | Hold Time | Pressure |
|---|---|---|---|---|
| Cycle Phase | (° C./Minute) | (° C.) | (Hours) | (mT) |
| Freezing | | | | — |
| Step 1 | 0.3 | −40 | 1 | |
| Step 2 | 0.3 | −5 | 2 | |
| Step 3 | 0.3 | −40 | 3 | |
| Total Ramp Time | | | 6.4 | |
| Primary Drying | 0.2 | −20 | 2.7 | 100 |
| Ramp Time | | | 1.7 | |
| Secondary Drying | 0.2 | 25 | 15 | 100 |
| Ramp Time | | | 3.75 | |

| | |
|---|---|
| Total Cycle Time | 62 |
| (Hours) | |

After completion of the lyophilization cycle, the product chamber is purged with sterile nitrogen gas immediately after breaking the vacuum. The vials are stoppered under nitrogen in the lyophilizer, crimped with aluminum seals, and then stored at 2-8° C.

The lyophilized composition is reconstituted with 0.6 mL of 12.5 mM sodium succinate diluent prior to injection. A succinate buffer was chosen as diluent for the peptides because succinate is already a compatible part of the peptide drug product formulation and it is the appropriate buffer to bring the final product into the desired pH range for clinical use. The final pH after reconstitution is between 4.2 and 5.2. In a particular embodiment, the final pH after reconstitution is about 4.7.

To enhance immunogenicity of the final injectable peptide composition, one or more suitable adjuvants can be used. Such adjuvants include, but are not limited to:

(1) aluminum salts (alum), such as aluminum hydroxide, aluminum phosphate, aluminum sulfate, etc.;

(2) oil-in-water emulsion formulations (with or without other specific immunostimulating agents such as muramyl peptides (see below) or bacterial cell wall components), such as, for example,

(a) MF59 (PCT Publ. No. WO 90/14837), containing 5% Squalene, 0.5% Tween 80, and 0.5% Span 85 (optionally containing various amounts of MTP-PE (see below, although not required)) formulated into submicron particles using a microfluidizer such as Model 110Y microfluidizer (Microfluidics, Newton, Mass.),

(b) SAF, containing 10% Squalene, 0.4% Tween 80, 5% pluronic-blocked polymer L121, and thr-MDP (see below) either microfluidized into a submicron emulsion or vortexed to generate a larger particle size emulsion, and

(c) Ribi™ adjuvant system (RAS), (Corixa, Hamilton, Mont.) containing 2% Squalene, 0.2% Tween 80, and one or more bacterial cell wall components from the group consisting of 3-O-deaylated monophosphorylipid A (MPL™) described in U.S. Pat. No. 4,912,094 (Corixa), trehalose dimycolate (TDM), and cell wall skeleton (CWS), preferably MPL+CWS (Detox™);

(3) saponin adjuvants, such as Quil A or STIMULON™ QS-21 (Antigenics, Framingham, Mass.) (U.S. Pat. No. 5,057,540) may be used or particles generated therefrom such as ISCOMs (immunostimulating complexes);

(4) bacterial lipopolysaccharides, synthetic lipid A analogs such as aminoalkyl glucosamine phosphate compounds (AGP), or derivatives or analogs thereof, which are available from Corixa, and which are described in U.S. Pat. No. 6,113,918; one such AGP is 2-[(R)-3-Tetradecanoyloxytetradecanoylamino]ethyl 2-Deoxy-4-O-phosphono-3-O-[(R)-3-tetradecanoyloxytetradecanoyl]-2-[(R)-3-tetradecanoyloxytetradecanoylamino]-b-D-glucopyranoside, which is also know as 529 (formerly known as RC529), which is formulated as an aqueous form or as a stable emulsion, synthetic polynucleotides such as oligonucleotides containing CpG motif(s) (U.S. Pat. No. 6,207,646);

(5) cytokines, such as interleukins (e.g., IL-1, IL-2, IL-4, IL-5, IL-6, IL-7, IL-12, IL-15, IL-18, etc.), interferons (e.g., gamma interferon), granulocyte magrophage colony stimulating factor (GM-CSF), macrophage colony stimulating factor (M-CSF), tumor nucrosis factor (TNF), etc.;

(6) detoxified mutants of a bacterial ADP-ribosylating toxin such as a cholera toxin (CT) either in a wild-type or mutant form, for example, where the glutamic acid at amino acid position 29 is replaced by another amino acid, preferably a histidine, in accordance with published international patent application number WO 00/18434 (see also WO 02/098368 and WO 02/098369), a pertussis toxin (PT), or an E. coli heat-labile toxin (LT), particularly LT-K63, LT-R72, CT-S109, PT-K9/G129 (see, e.g., WO 93/13302 and WO 92/19265); and

(7) other substances that act as immunostimulating agents to enhance the effectiveness of the composition.

As mentioned above, muramyl peptides include, but are not limited to, N-acetyl-muramyl-L-threonyl-D-isoglutamine (thr-MDP), N-acetyl-normuramyl-L-alanine-2-(1′-2′ dipalmitoyl-sn-glycero-3-hydroxyphosphoryloxy)-ethylamine (MTP-PE), etc.

In one embodiment, the final injectable HIV peptide composition contains four Th/CTL lyophilized peptides (Peptides A, B, C and J), adjuvanted with RC529-SE and GM-CSF. The injectable composition is prepared in the following manner. 0.6 ml sodium succinate diluent is injected into the vial of lyophilized peptides. After gentle swirling, 0.15 ml of RC529-SE is added to the reconstituted peptide vial. Then 0.75 ml of GM-CSF is added to the vial. After gentle mixing, the contents of the vial are drawn into the syringe. One ml is administered intramuscularly to the subject.

### Stability

The HIV tetravalent peptide composition (Peptides A, B, C and J), formulated at varying concentrations (1 mg/ml, 625 μg/mL, 187.5 μg/mL of each peptide), as well as monovalent at 1 mg/mL, was held for stability at 2-8° C. and 40° C. The stability was also monitored for pre-lyophilization formulations. Stability assays basically include appearance (visual inspection), pH, FLS, peptide fluorescence, and RP-HPLC for peptide quantification. The stability specifications are provided in Table 3 below.

TABLE 3
Specifications for Stability

| Tests | Specifications | Notes |
|---|---|---|
| Cake | White cake without any | Test performed on lyophilized |
| Appearance | meltback or contraction | cakes |
| Appearance | Clear to turbid | Following reconstitution with |
| | colorless solution | 12.5 mM sodium succinate |
| Reconstitution | <2 minutes | |
| Time | | |
| pH | 4.7 ± 0.5 | Following reconstitution with |
| | | 12.5 mM sodium succinate |
| Identity for | Retention time of sample | Following reconstitution with |
| CTL Peptides | matches the retention | 12.5 mM sodium succinate |
| | time of the standard | |
| | within ±10% | |
| Moisture | <3% | Test performed on lyophilized |
| | | cakes |
| Potency | 469-781 μg/mL for | Following reconstitution with |
| | each peptide | 12.5 mM sodium succinate |
| Sterility | Sterile | Tested at 0, 12, 24 and |
| | | 36-month time points |

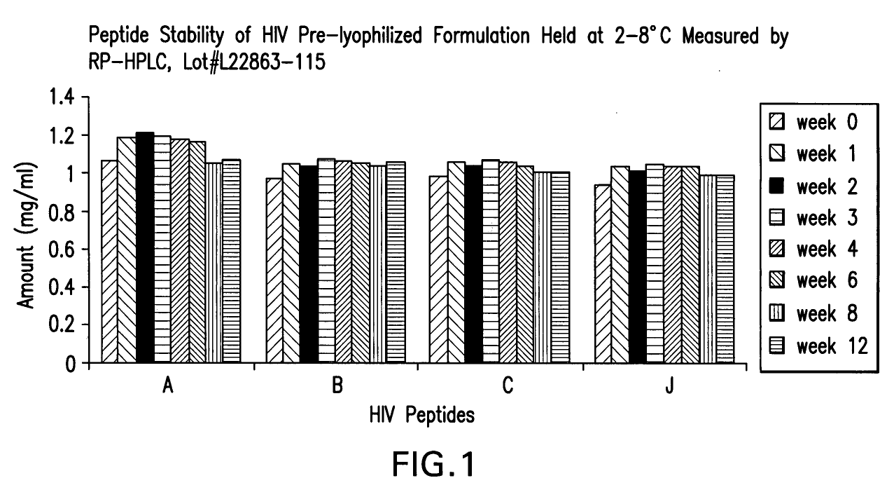

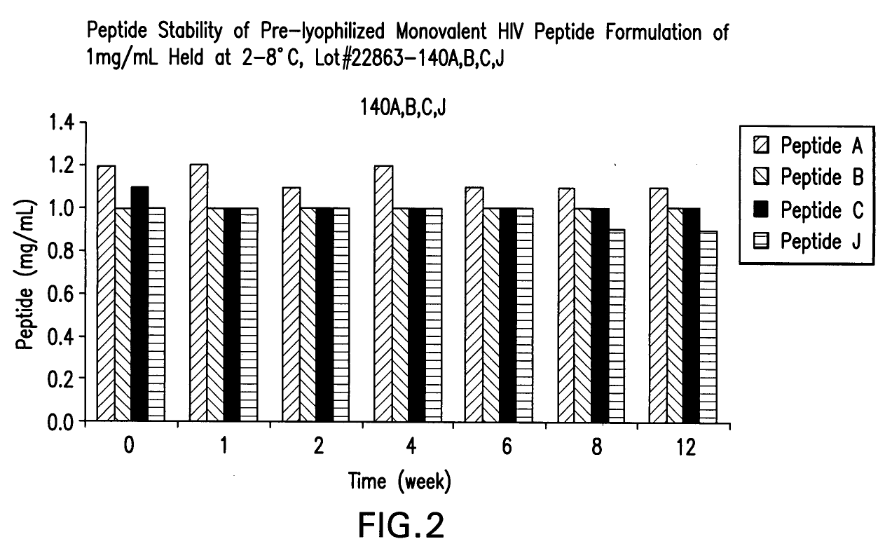

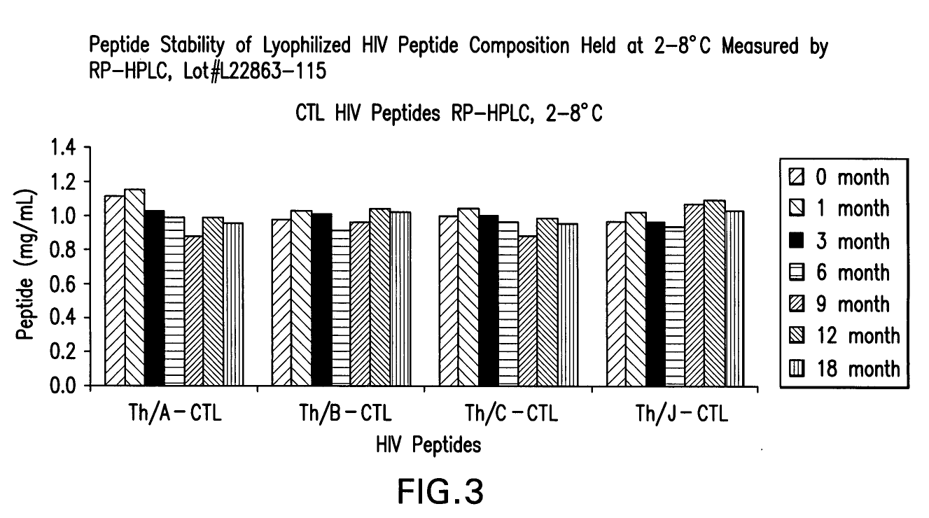

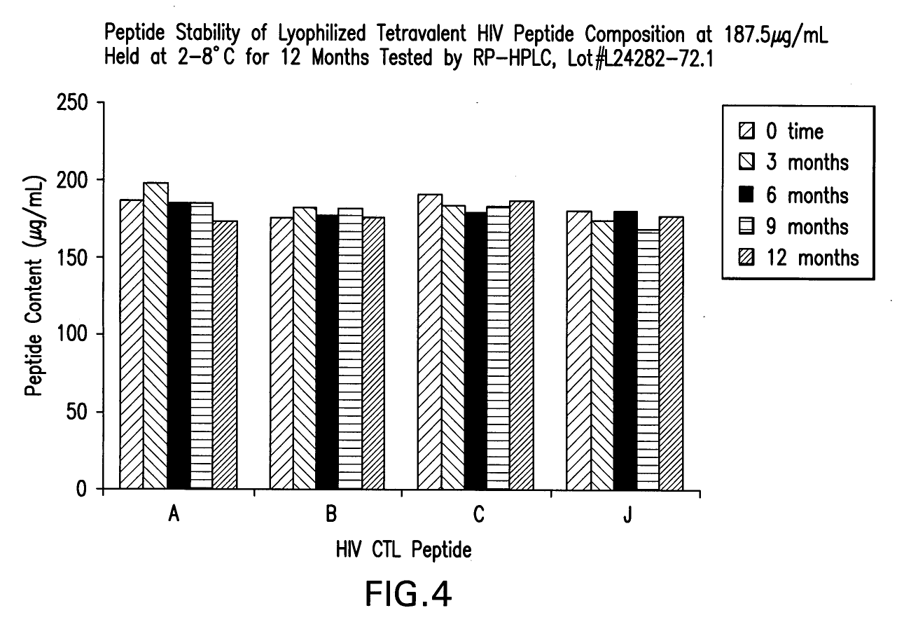

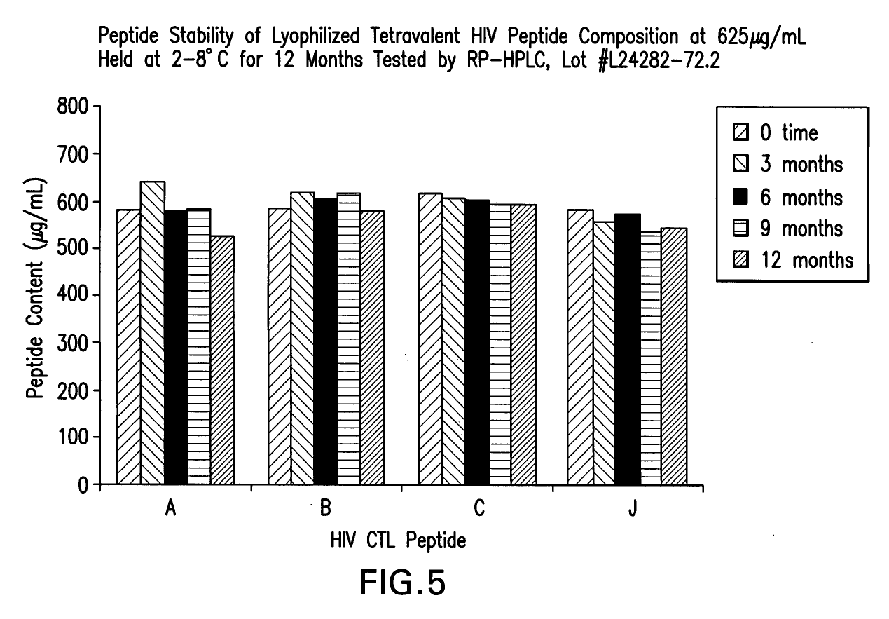

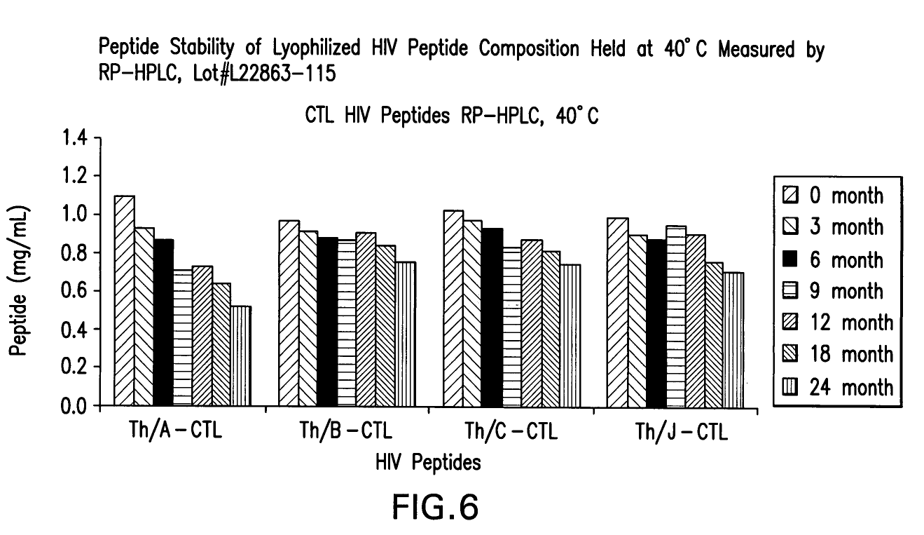

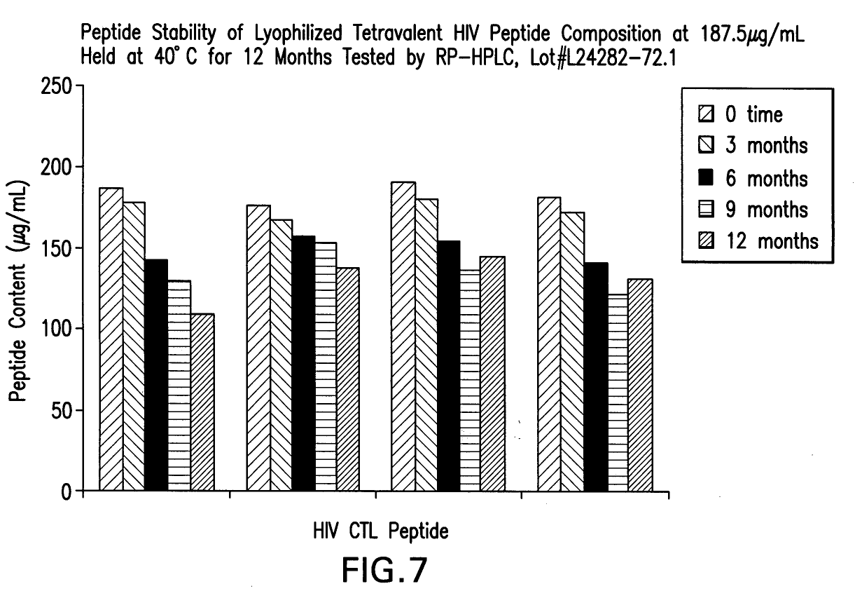

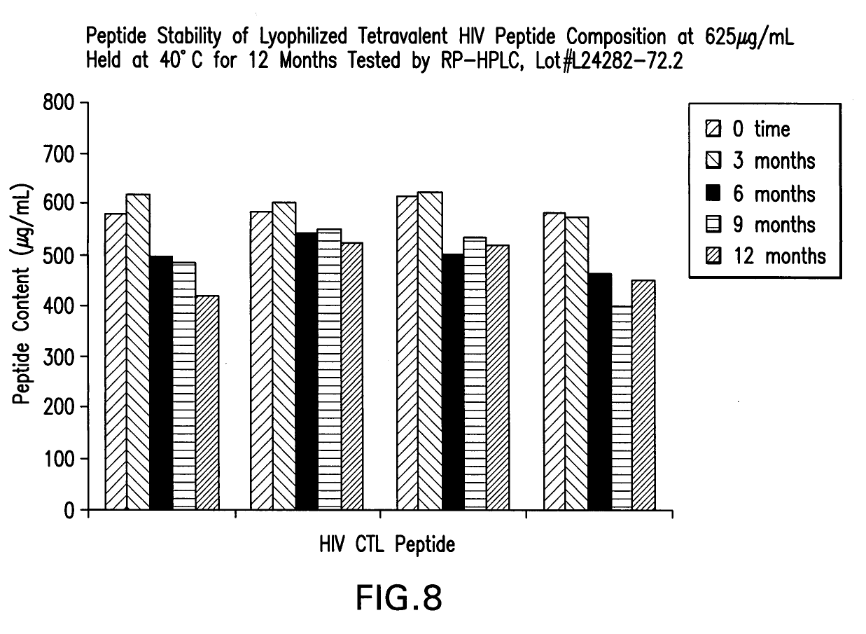

The pH was stable for all formulations at both 2-8° C. and 40° C. The formulated bulk (pre-lyophilization formulation) of 1 mg/mL of each peptide was stable up to 12 weeks at 2-8° C. based on RP-HPLC (FIGS. 1 and 2). The lyophilized tetravalent peptides (Peptides A, B, C and J) at 1 mg/mL each were stable at 2-8° C. for 24 months (FIG. 3). The lyophilized tetravalent peptides formulated at 187.5 μg/mL and 625 μg/mL were stable at 2-8° C. for at least 12 months based on the available data (FIGS. 4 and 5). At the accelerated temperature of 40° C., potency decreases (mainly Peptides A and J) were observed in all three lyophilized tetravalent formulations at the six-months time point (FIGS. 6-8).

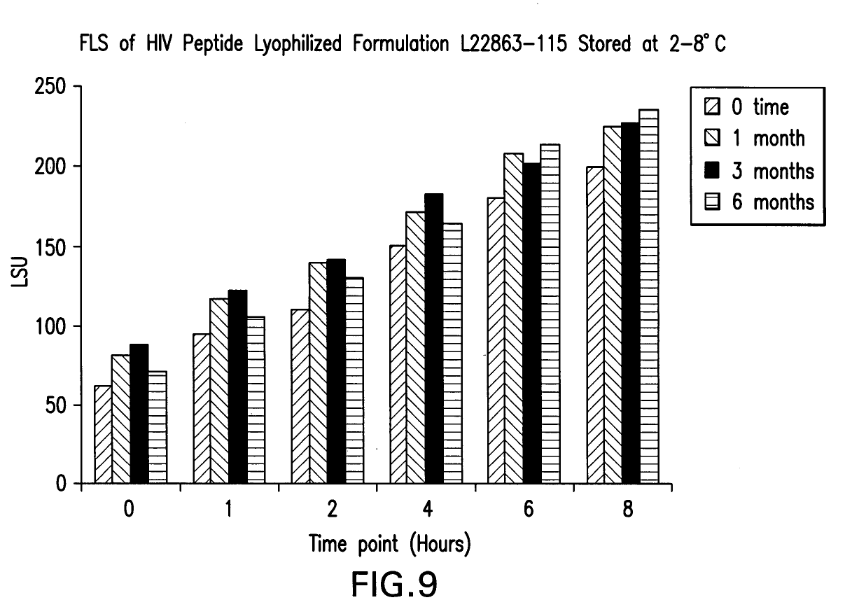

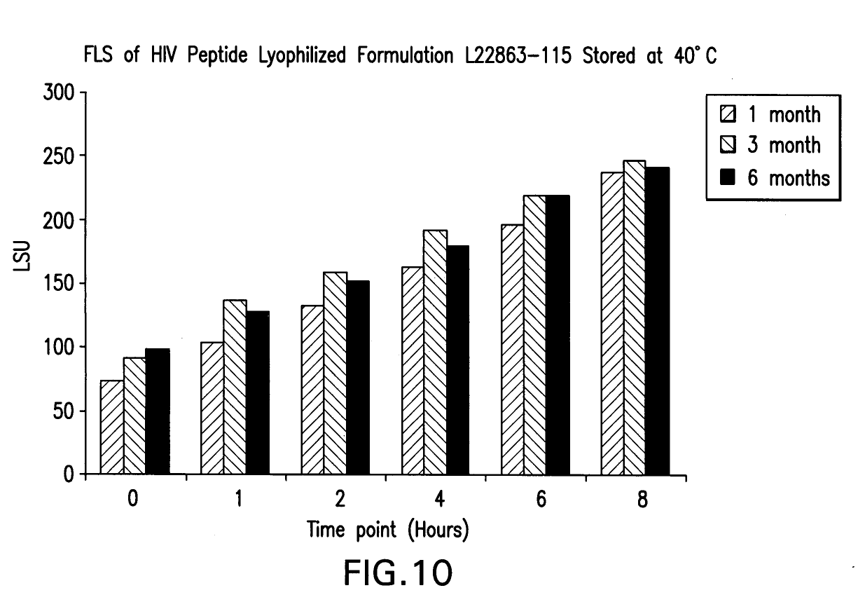

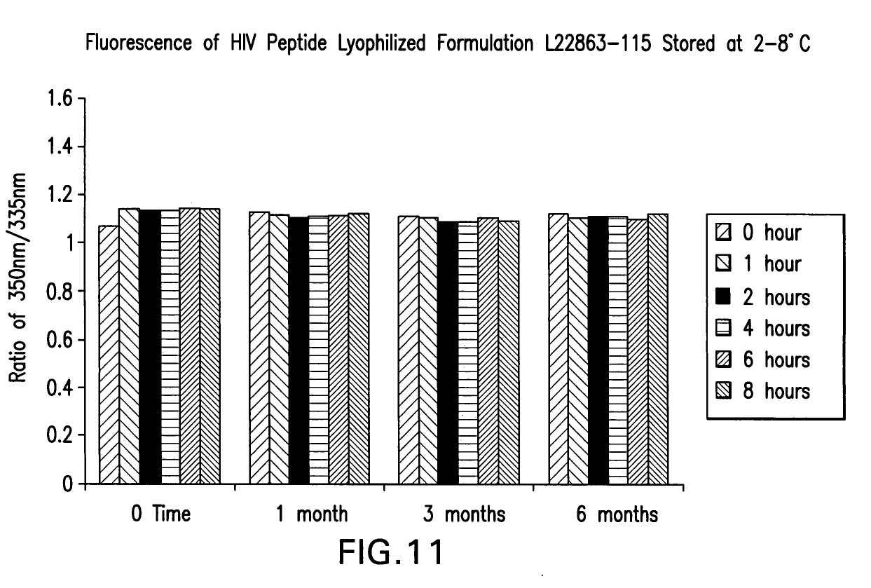

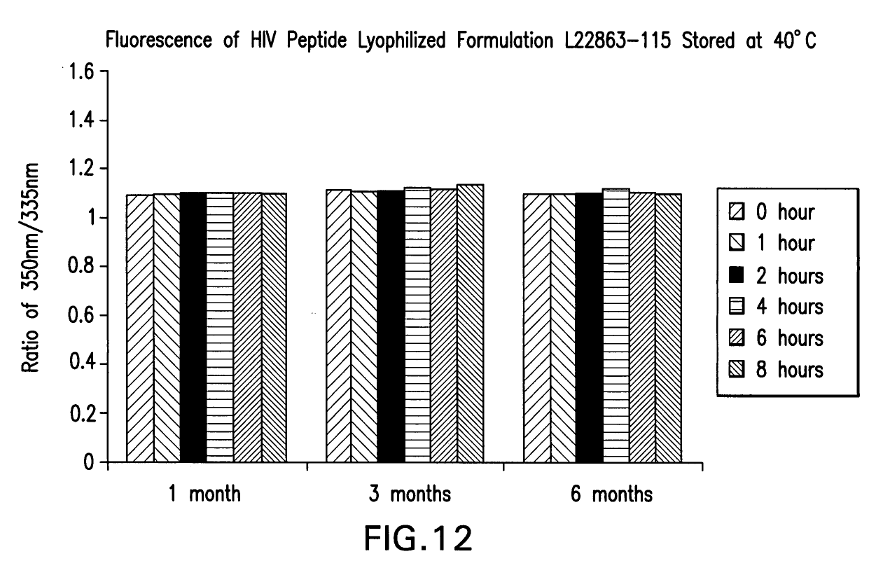

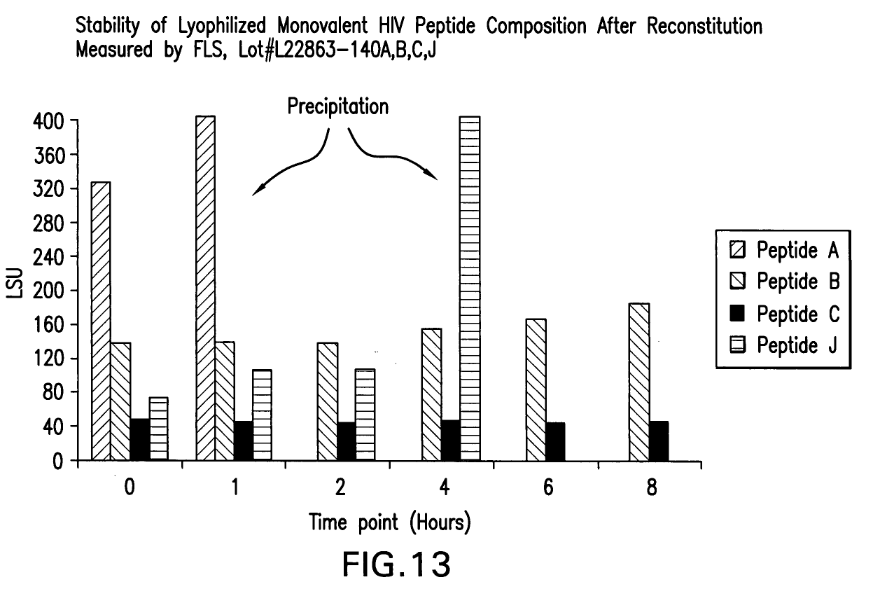

For short-term stability following reconstitution with sodium succinate diluent, fluorescence light scattering (FLS) and peptide fluorescence data show that the lyophilized tetravalent peptides at 1 mg/mL of each peptide, were stable for eight hours (FIGS. 9-12). It was also observed that lyophilized monovalent Peptides A and J precipitated out of solution within four hours after reconstitution (FIG. 13).

### EXAMPLES

The above disclosure generally describes the present invention. A more complete understanding can be obtained by reference to the following specific examples. These examples are described solely for the purpose of illustration and are not intended to limit the scope of the invention.

### Abbreviations

CTL Cytotoxic T Lymphocyte
GM-CSF Granulocyte Macrophage Colony Stimulating Factor
HIV Human Immunodeficiency Virus
mT Millitorr
PPL PolyPeptide Labs
PVDF Polyvinylidene Fluoride
QS Quantum Sufficient
RC529-SE RC-529 Stable Emulsion
RP-HPLC Reverse Phase High Pressure Liquid Chromatography
TFA Trifluoroacetic Acid
Th/CTL T helper Cytotoxic T Lymphocyte
WFI Water for Injection

### Raw Materials Used for the HIV Th/CTL Peptide Composition

| Raw Material | Source | Function |
|---|---|---|
| Th/A-CTL Acetate | Polypeptide Laboratories | Active ingredient |
| Th/B-CTL Acetate | Polypeptide Laboratories | Active ingredient |
| Th/C-CTL Acetate | Polypeptide Laboratories | Active ingredient |
| Th/J-CTL | Polypeptide Laboratories | Active ingredient |
| Trifluoroacetate | | |
| D-Mannitol | EM Science | Excipient |
| Succinic Acid | Mallinckrodt Chemicals | Buffer |
| Hydrochloric Acid | EM Science | To adjust pH |
| Water for Injection | Cardinal Health | Diluent |
| Liquid Nitrogen | Praxair, Inc. | Processing aid |
| Millipak 20 Durapore/ | Millipore | Sterile filtration |
| PVDF filter | | |

### Example 1

### Preparation of Formulation Buffer to Dissolve HIV Th/CTL Peptides

1.5609 kg of WFI USP/EP, which is equivalent to 70% of the final weight of buffer solution were added to a 4L, dedicated, tared compounding vessel. With gentle mixing, 66.9 g of D-mannitol were added to the compounding vessel. The weighing container was rinsed three times with a portion of WFI (not exceeding 5% of the final volume). The buffer solution was mixed for 5 minutes. With gentle mixing, 3.3 g of succinic acid were added to the compounding vessel. The weighing container was rinsed three times with a portion of WFI (not exceeding 5% of the final volume). The buffer solution was mixed for 5 minutes. Measured pH was 2.9. Three percent HCl (v/v) was added dropwise to adjust the pH to 1.7. (A total of 66.5 mL 3% HCl was added.) The buffer solution was mixed for 5 minutes, and then filtered through a 0.2 Millipak 20 Durapore/PVDF filter into a dedicated compounding vessel. The filtered buffer solution was stored ≦24 hours at 2-8° C.

### Example 2

### Formulation, Fill and Lyophilzation of HIV Th/CTL Peptides

Formulation and Fill. The following amounts of HIV Th/CTL peptides were weighed out: Peptide A: 1.357 g, Peptide B: 1.324 g, Peptide C: 1.401 g, Peptide J: 1.321 g. 1.5204 kg of filtered buffer solution (equivalent to 75% of the final batch weight) was transferred to a dedicated, tared, primary compounding vessel. With gentle mixing, HIV peptides A, B, C and J were transferred sequentially to the primary compounding vessel. The weighing container was mixed three times with a portion of filtered buffer solution (not exceeding 5% of the final batch volume). The peptide formulation was mixed gently for five minutes, QS'd to final batch weight using the 0.2 μm filtered buffer solution, and then mixed gently again for five minutes. Measured pH was 2.0. Visual inspection of the bulk: clear and colorless solution. After filtration through a flushed (using WFI) 0.2 μM Millipak 20 Durapore/PVDF filter, the formulated bulk was filled into glass vials with a fill volume of 0.63±0.03 mL (Target 0.60 mL).

Lyophilization. The formulated bulk-containing vials were loaded on a shelf already maintained at 5° C. and equilibrated for two hours before freezing to −40° C. The condenser temperature was <−70° C. The primary drying was conducted at −20° C. with 100 mT vacuum with inclusion of annealing and thermal treatment in the freezing phase. Secondary drying was conducted at 25° C. with 100 mT vacuum as well. The details of the lyophilization cycle are recorded in Table 2 above.

After completion of the lyophilization cycle, the product chamber was purged with sterile nitrogen gas immediately after breaking the vacuum. The vials were stoppered under nitrogen in the lyophilizer, crimped with aluminum seals, and then stored at 2-8° C.

### Example 3

### Formulation of the Final Injectable Material

The final injectable HIV peptide composition containing the four Th/CTL lyophilized peptides (Peptides A, B, C and J), was adjuvanted with RC529-SE and GM-CSF. The injectable composition was prepared in the following manner. 0.6 ml of 12.5 mM sodium succinate diluent was injected into the vial of lyophilized peptides. vial. Then 0.75 ml of GM-CSF was added to the vial. After gentle mixing, the contents of the vial were drawn into the syringe. One ml was administered intramuscularly to the subject.

It should be understood that the foregoing discussion and examples merely present a detailed description of certain embodiments. It therefore should be apparent to those of ordinary skill in the art that various modifications and equivalents can be made without departing from the spirit and scope of the invention.

All journal articles, other references, patents and patent applications that are identified in this patent application are incorporated by reference in their entirety.

What is claimed is:

1. A process for solubilizing hydrophobic HIV Th/CTL peptides, comprising the steps of:
(a) preparing an aqueous formulation buffer comprising 12.5 mM succinic acid and 3% mannitol;
(b) adjusting the pH of the formulation buffer to 1.7 to 2.1; and
(c) dissolving at least one hydrophobic HIV Th/CTL peptide in the formulation buffer.

2. The process of claim 1, wherein a mixture of at least four hydrophobic HIV Th/CTL peptides are dissolved in the formulation buffer.

3. The process of claim 2, wherein the peptide mixture comprises HIV A-Th/A-CTL (SEQ ID NO:1), B-Th/B-CTL (SEQ ID NO:2), C-Th/C-CTL (SEQ ID NO:3) and A*-Th/J-CTL (SEQ ID NO:4).

4. The process of claim 2, wherein the peptide mixture comprises HIV A-Th/A-CTL (SEQ ID NO:1), B-Th/B-CTL (SEQ ID NO:2), C-Th/C-CTL (SEQ ID NO:3), A*Th/J-CTL (SEQ ID NO:4), A*-Th/L-CTL (SEQ ID NO:5), A*-Th/M1-CTL (SEQ ID NO:6), A*-Th/M2-CTL (SEQ ID NO:7) and A*-Th/R-CTL (SEQ ID NO:8).

5. A process for preparing a stable formulation of hydrophobic HIV Th/CTL peptides, comprising the steps of:
(a) preparing an aqueous formulation buffer comprising 12.5 mM succinic acid and 3% mannitol, and adjusting the pH of the formulation buffer to 1.7 to 2.1;
(b) dissolving a mixture of hydrophobic HIV Th/CTL peptides in the formulation buffer;
(c) filtering the peptide formulation obtained in step (b); and
(d) lyophilizing the filtrate, whereby the lyophillized HIV Th/CTL peptides are stable at 2-8° C. for at least 24 months.

6. The process of claim 4, wherein the mixture of hydrophobic HIV Th/CTL peptides comprises A-Th/A-CTL (SEQ ID NO:1), B-Th/B-CTL (SEQ ID NO:2), C-Th/C-CTL (SEQ ID NO:3) and A*-Th/J-CTL (SEQ ID NO:4).

7. The process of claim 4, wherein the mixture of hydrophobic HIV Th/CTL peptides comprises A-Th/A-CTL (SEQ ID NO:1), B-Th/B-CTL (SEQ ID NO:2), C-Th/C-CTL (SEQ ID NO:3), A*Th/J-CTL (SEQ ID NO:4), A*-Th/L-CTL (SEQ ID NO:5), A*-Th/M1-CTL (SEQ ID NO:6), A*-Th/M2-CTL (SEQ ID NO:7) and A*-Th/R-CTL (SEQ ID NO:8).

8. A process for preparing an immunogenic composition for the prevention or treatment of HIV, comprising: reconstituting the lyophilized HIV Th/CTL peptides of claim 4 with 12.5 mM sodium succinate prior to use, wherein the pH after reconstitution is 4.2 to 5.2, and mixing the reconstituted peptides with at least one adjuvant to produce an immunogenic composition that is stable at 2-8° C. for at least three hours.

9. The process of claim 8, wherein the adjuvant is selected from the group consisting of GM-CSF and RC 529-SE, or a combination thereof.

10. A lyophilized composition comprising hydrophobic HIV Th/CTL peptides that are stable at 2-8° C. for at least 24 months, wherein the peptides are produced by the process of claim 5.
